(19) United States
(12) Patent Application Publication
(10) Pub. No.: US 2017/0209352 A1
(43) Pub. Date: Jul. 27, 2017

(54) METHOD FOR THE SIMULTANEOUS PERMANENT SHAPING AND DYEING OF KERATINIC FIBERS

(71) Applicant: Henkel AG & Co. KGaA, Dusseldorf (DE)

(72) Inventors: Torsten Lechner, Dusseldorf (DE); Yvonne Lissner, Hamburg (DE); Birgit Rautenberg-Groth, Ellerau (DE)

(73) Assignee: Henkel AG & Co. KGaA, Dusseldorf (DE)

(21) Appl. No.: 15/482,166

(22) Filed: Apr. 7, 2017

Related U.S. Application Data
(63) Continuation of application No. PCT/EP2015/070359, filed on Sep. 7, 2015.

(30) Foreign Application Priority Data
Oct. 15, 2014 (DE) 102014220919.6

(51) Int. Cl. A61K 8/41; A61Q 5/08; A61K 8/60; A61K 8/19; A61K 8/04; A61K 8/42; A61K 8/86; A61K 8/06; A61K 8/34; A61Q 5/04; A61K 8/92

(52) U.S. Cl. CPC A61K 8/41; A61Q 5/04; A61Q 5/08; A61K 8/602; A61K 8/19; A61K 8/922; A61K 8/42; A61K 8/86; A61K 8/06; A61K 8/411; A61K 8/347; A61K 8/046; A61K 2800/884; A61K 2800/48; A61K 2800/596

(57) ABSTRACT

A method for the permanent shaping and color modification of keratinic fibers, in a single process, includes the following method steps in sequence:
deforming keratinic fibers, using deformation aids,
applying an aqueous composition, including at least one keratin-reducing compound and at least one alkalizing agent, to the keratinic fibers situated on the deformation aids, and leaving this composition for a period of 5 to 50 minutes,
rinsing the keratinic fibers situated on the deformation aids,
applying a composition, including at least one oxidation dye precursor, at least one oxidizing agent, at least one alkalizing agent, and at least one surfactant, as foam from an applicator to the keratinic fibers situated on the deformation aids, and leaving this composition for a period of 10 to 45 minutes,
removing the deformation aids from the keratinic fibers and rinsing the keratinic fibers, and
optionally applying an aftertreatment agent to the keratinic fibers.

### FIELD OF THE INVENTION

The present invention generally relates to a method for the permanent shaping and color modification of keratinic fibers, in particular human hair, in a single process.

### BACKGROUND OF THE INVENTION

Permanent deformation of keratin-containing fibers is usually carried out in such a way that the fiber is mechanically deformed, and the deformation is fixed using suitable aids. Before and/or after this deformation, the fiber is treated with a keratin-reducing preparation. After a rinsing operation, the fiber is then treated with an oxidizing agent preparation in the so-called fixing step, rinsed, and the deformation aids (curlers, papillotes) are removed during or after the fixing step. When a mercaptan, for example ammonium thioglycolate, is used as the keratin-reducing component, the mercaptan cleaves a portion of the disulfide bridges of the keratin molecule to form thiol groups, resulting in softening of the keratin fiber or swelling of the fibers, with enlargement of the fiber diameter. During the subsequent oxidative fixing, disulfide bridges are re-linked in the keratin of the hair, so that the keratin structure is fixed in the specified deformation. Alternatively, it is known to use sulfite instead of the mercaptans for the hair deformation. By use of hydrogen sulfite solutions and/or sulfite solutions and/or disulfite solutions, disulfide bridges of the keratin are cleaved in a sulfitolysis process according to the equation

R—S—S—R+HSO₃⁽⁻⁾→R—SH+R—S—SO₃⁽⁻⁾,

thus achieving softening of the keratin fiber. Reducing agents including hydrogen sulfite, sulfite, or disulfite do not have the strong inherent odor of the mercaptan-containing agents. The cleavage, as described above, may be reversed in a fixing step, using an oxidizing agent, to form new disulfide bridges.

When dyeing of the keratinic fiber in addition to the shaping is also desired, the dyeing may be carried out as a separate treatment before or after the shaping that takes place. However, in particular in the case of oxidative dyeing, this results in extreme stress on the keratinic fibers, since each oxidative treatment of the fibers damages their internal structure. In addition, such an operation is very time-consuming, since a period of two weeks or more must be provided between the shaping and the dyeing treatment in order to avoid the above-described extreme stress and accompanying damage. For this reason, several methods for simultaneously shaping and dyeing keratinic fibers, in particular hair, have already been proposed. In many cases, for this purpose an oxidizing agent preparation that includes substantive dyes and/or oxidation dye precursors in addition to the oxidizing agent is used in the fixing step. Such a procedure is described in DE 19713698, for example. However, this procedure has the disadvantage that the dyeing takes place at the same time as the fixing, i.e., at a time when the fibers to be treated are placed on deformation aids and are thus under mechanical tension. This hinders the uniform application of the dye, so that there is a risk of a nonuniform dyeing result.

Methods for simultaneously shaping and dyeing of hair are known from EP 0352375 and EP 1287812, in which a keratin-reducing preparation that already includes the necessary substantive dyes and/or oxidation dye precursors is used. At least a portion of the particular keratin-reducing preparation is applied to the hair after it has been mechanically deformed. However, the substantive dyes and/or oxidation dye precursors used for the dyeing do not always have satisfactory stability with respect to the keratin-reducing preparation, so that nonuniform shaping and dyeing results may occur in the event that the substantive dyes and/or oxidation dye precursors react with the keratin-reducing preparation.

It is therefore desirable to provide a method for shaping and dyeing keratinic fibers, in particular human hair, in which the shaping and the dyeing may be carried out in a single process, and which gives a comparable or better shaping result, uniformly dyes the keratinic fibers in the desired tint, and results in little or even no damage to the hair. Furthermore, other desirable features and characteristics of the present invention will become apparent from the subsequent detailed description of the invention and the appended claims, taken in conjunction with this background of the invention.

### BRIEF SUMMARY OF THE INVENTION

A first subject matter of the invention therefore relates to a method for the permanent shaping and color modification of keratinic fibers, in particular human hair, in a single process, the method comprising the following method steps in the stated sequence:
(a) deforming keratinic fibers, using deformation aids,
(b) applying an aqueous composition (M1), including at least one keratin-reducing compound and at least one alkalizing agent, to the keratinic fibers situated on the deformation aids, and leaving this composition (M1) on the keratinic fibers, situated on the deformation aids, for a period of 5 to 50 minutes,
(c) rinsing the keratinic fibers situated on the deformation aids,
(d) applying a composition (M2), including at least one oxidation dye precursor, at least one oxidizing agent, at least one alkalizing agent, and at least one surfactant, as foam from an applicator to the keratinic fibers situated on the deformation aids, and leaving this composition (M2) on the keratinic fibers, situated on the deformation aids, for a period of 10 to 45 minutes,
(e) removing the deformation aids from the keratinic fibers and rinsing the keratinic fibers, and
(f) optionally applying an aftertreatment agent to the keratinic fibers.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has surprisingly been found that that the object is achieved by a method in which, after applying deformation aids, the keratinic fibers are deformed by means of reducing agent composition, and a dye composition is subsequently applied. The method according to the invention results in an excellent shaping result as well as uniform and intensive dyeing or lightening. In addition, when the method procedure according to the invention is used, there is surprisingly little or no damage to the hair. Carrying out the shaping step and the dyeing step in succession allows significant time savings compared to the shaping and dyeing in two separate processes.

In principle, all animal hair, for example wool, horsehair, angora hair, fur, feathers, and products or textiles produced therefrom, may be used as keratin-containing fibers. However, the invention is preferably implemented within the scope of simultaneous hair shaping and dyeing, in particular permanent waving and dyeing of straight hair and wigs made therefrom.

According to the invention, the permanent deformation and color modification, i.e., the permanent wave and oxidative hair dyeing, is carried out in a single process. In this regard, “in a single process” means that there is a period of 30 seconds to 2 hours, preferably 30 seconds to 1 hour, more preferably 30 seconds to 30 minutes, in particular 30 seconds to 15 minutes, between the end of one method step and the beginning of the next method step.

Within the meaning of the method according to the invention, deformation aids are preferably so-called permanent wave curlers or papillotes.

Particularly preferred methods according to the invention result in waving of the keratinic fibers with simultaneous lightening or color modification of the hair color that is present before the method according to the invention is carried out. Therefore, a permanent wave is preferably carried out as permanent shaping, and lightening or dyeing is preferably carried out as color modification.

In the first method step (method step a) of the method according to the invention, the keratinic fibers are deformed, using deformation aids. In this regard, in particular permanent wave curlers or papillotes are suited as deformation aids. To facilitate placing the keratinic fibers on the deformation aids, it may be preferable according to the invention for the keratin-containing fibers to be moistened with water or washed with a hair cleaning agent prior to method step a). Using a hair cleaning agent, in particular a hair shampoo, may be advantageous when the hair is very dirty. After rinsing out the hair shampoo, the hair is then rubbed with a towel so that perceptible residual moisture remains in the hair. If the hair is not very dirty, it is preferable to moisten the keratinic fibers with water to ensure separation into defined, individual strands of hair. This may take place, for example, by spraying the fibers with a liquid, preferably water.

To avoid excessive stress and damage to the keratinic fibers while carrying out the method according to the invention, deformation aids having a specific diameter are preferably used in method step a). Particularly preferred methods according to the invention are therefore characterized in that the deformation aids used in method step a) have a diameter of 1 to 10 cm, preferably 1 to 8 cm, more preferably 1 to 6 cm, in particular 2 to 5 cm.

In a second method step (method step b) an aqueous composition (M1) is applied to the keratinic fibers already situated on the deformation aids. This aqueous composition (M1), also referred to below as reducing agent, is left on the keratinic fibers for a period of 5 to 50 minutes. According to the invention, however, fairly short exposure times to the reducing agent are preferred. Particularly preferred methods according to the invention are therefore characterized in that the composition (M1) used in method step b) is left on the keratinic fibers situated on the deformation aids for a period of 10 to 50 minutes, preferably 10 to 45 minutes, more preferably 10 to 40 minutes, in particular 20 to 40 minutes. Due to the use of a reducing agent (M1), a portion of the disulfide bridges of the keratin molecule is reduced to form thiol groups, resulting in softening of the keratin fibers. To obtain a uniform shaping result, in particular a permanent wave result, the reducing agent (M1) should be applied uniformly to the keratinic fibers on the deformation aids. For this purpose, it may be preferable to repeat the application of the reducing agent several times to ensure that the keratinic fibers are completely wetted with the reducing agent (M1).

Following the exposure time to the reducing agent, the keratinic fibers on the deformation aids are rinsed out with water (method step c).

After rinsing the keratinic fibers, in method step d) of the method according to the invention a composition (M2), also referred to below as a coloring agent, is applied to the keratinic fibers, which are still situated on the deformation aids, and allowed to act for a period of 25 to 45 minutes. Methods according to the invention are therefore characterized in that the composition (M2) used in method step d) is left on the keratinic fibers for a period of 10 to 40 minutes, preferably 20 to 40 minutes, in particular 25 to 35 minutes. To ensure uniform, complete wetting of the keratinic fibers, it may be preferable to apply coloring agent to the keratinic fibers several times in succession. The oxidizing agent, in particular hydrogen peroxide, present in the coloring agent on the one hand results in partial oxidation of the thiol groups of the softened keratinic fibers, and thus, prefixing of the shaped, in particular waved, keratinic fibers. On the other hand, the oxidizing agent in the composition (M2) results in formation of the desired color from the oxidation dye precursors, in particular lightening or dyeing of the keratinic fibers.

After the deformation aids are removed composition (M2) is rinsed out in method step e) of the method according to the invention, shaping, in particular waving, as well as color modification, in particular lightening or dyeing, of the keratinic fibers is directly obtained without having to carry out a further oxidative dyeing operation, and without excessively damaging the keratinic fibers due to the simultaneous waving and lightening or dyeing. Considerable time savings may thus be achieved compared to carrying out the waving and lightening or dyeing as two separate processes, since there may typically be a certain period of 1 to 3 weeks between these processes in order to avoid damage to the hair. Within the scope of the method according to the invention, it is preferable to use water having a temperature of 20 to 45° C. for the rinsing of the keratinic fibers carried out in method step e).

The aqueous composition (M1) used in method step b) is a reducing agent that includes at least one keratin-reducing compound. According to the invention, an aqueous composition is understood to mean a composition that includes at least 50% by weight water, based on the total weight of the composition. This aqueous composition (M1) may be present in various forms, for example as a lotion, oil-in-water emulsion, or water-in-oil emulsion. According to the invention, the composition (M1) used as a keratin-reducing compound in method step b) preferably includes at least one compound from the group comprising thioglycolic acid, thiolactic acid, thiomalic acid, phenylthioglycolic acid, mercaptoethanesulfonic acid, and the salts and esters thereof, cysteamine, cysteine, Bunte salts and salts of sulfurous acid, alkali disulfites, for example sodium disulfite (Na₂S₂O₅) and potassium disulfite (K₂S₂O₅), and magnesium disulfite and ammonium disulfite ((NH₄)₂S₂O₅), hydrogen sulfites as alkali, magnesium, ammonium, or alkanolammonium salts based on a C₂-C₄ mono-, di-, or trialkanolamine, and sulfites as alkali, ammonium, or alkanolammonium salts based on a C₂-C₄ mono-, di-, or trialkanolamine. The above-mentioned compounds are able to reduce the disulfide bridges of the keratin to form thiol groups, and thus to ensure the softening of the keratin fibers necessary for the shaping.

Within the scope of this embodiment, it has proven to be particularly advantageous when the composition (M1) used as a keratin-reducing compound in method step b) includes at least one compound from the group comprising thioglycolic acid, thiolactic acid, and cysteine, and the salts thereof. Using the above-mentioned keratin-reducing compounds ensures a sufficient reduction of the disulfide bridges at relatively low use concentrations, so that the development of unpleasant odors during the shaping may be largely avoided.

According to the invention, the composition (M1) used in method step b) preferably includes the at least one keratin-reducing compound in an overall quantity of 5 to 20% by weight, preferably 7 to 18% by weight, more preferably 9 to 16% by weight, in particular 10 to 15% by weight, based on the total weight of the aqueous composition (M1). Using such quantities ensures sufficient softening of the keratin fibers, but without excessively damaging the fibers or releasing unpleasant odors during the application. A good shaping result without excessive damage to the hair may thus be achieved.

The reducing agents (M1) used in method step b) also include at least one alkalizing agent for setting the desired pH and for assisting with hair swelling, i.e., enlargement of the hair diameter. The composition (M1) used in method step b) preferably includes as alkalizing agent at least one compound from the group comprising sodium hydroxide, potassium hydroxide, ammonium hydroxide, ammonia, monoethanolamine, 2-amino-2-methylpropane, and alkali and ammonium hydrogen carbonates. These alkalizing agents are stable even in the presence of the reducing compound(s), and do not result in instability or pH fluctuations of the reducing agents (M1).

In this regard, it is advantageous when the composition (M1) used in method step b) includes ammonium hydrogen carbonate and/or ammonium hydroxide as alkalizing agent. Use of these alkalizing agents has proven to be particularly advantageous with regard to the pH stability and storage stability of the reducing agents (M1).

According to one embodiment of the present invention, the composition (M1) used in method step b) includes the at least one alkalizing agent in an overall quantity of 0.1 to 15% by weight, preferably 0.5 to 12% by weight, more preferably 1.0 to 10% by weight, in particular 1.5 to 7% by weight, based on the total weight of the aqueous composition (M1). Use of the above-mentioned quantities results in superior assistance in hair swelling. In addition, the setting of the desired pH values of pH 5 to pH 12 is ensured when these quantities are used.

Within the scope of the present invention, compositions (M1) preferably used in method step b) therefore have a pH of 5 to 12, preferably 5 to 10, in particular 5 to 9.5, at 20° C.

Particularly good results are obtained within the scope of the present invention when the composition (M1) used in method step b) has a weight ratio of the keratin-reducing compound to the alkalizing agent of 1:200 to 1:1, preferably 1:50 to 1:1, more preferably 1:30 to 1:1, very preferably 1:20 to 1:1, in particular 1:10 to 1:1. Use of the above-mentioned weight ratios results in particularly effective hair softening and hair swelling, and thus ensures a long-lasting shaping result that also is not significantly influenced, in particular impaired, by the subsequent dyeing step, in particular the lightening or dyeing step.

The aqueous composition (M1) may include further active substances and ingredients in addition to the above-mentioned ingredients. The composition (M1) used in method step b) preferably additionally includes at least one further compound selected from the group of (i) surfactants; (ii) cationic polymers; (iii) protein hydrolysates; (iv) oils; (v) thickeners; and (vi) the mixtures thereof.

Within the meaning of the present invention, surfactants are amphiphilic (bifunctional) compounds composed of at least one hydrophobic and at least one hydrophilic molecular portion. A basic property of surfactants and emulsifiers is the oriented absorption to boundary surfaces, and the aggregation into micelles and the formation of lyotrophic phases. Within the scope of the present invention, usable surfactants are selected from the group of nonionic surfactants, anionic surfactants, amphoteric surfactants, zwitterionic surfactants, cationic surfactants, and the mixtures thereof.

According to the invention, reducing agents (M1) are particularly preferably used in the method according to the invention which additionally include at least one nonionic surfactant from the group comprising (i) alkylene oxide addition products with alcohols having 8 to 30 carbon atoms or carboxylic acids having 8 to 30 carbon atoms, which include 2 to 30 moles of ethylene oxide per mole of alcohol or carboxylic acid, respectively; (ii) carboxylic acid esters of ethoxylated and/or propoxylated glycerin having 8 to 30 carbon atoms in the carboxylic acid chain and 1 to 30 moles of ethylene oxide and/or propylene oxide per mole of glycerin; (iii) alkyl polyglucosides of formula R¹O-[G]ₚ, where R¹ stands for an alkyl and/or alkenyl functional group including 4 to 22 carbon atoms, G stands for a sugar functional group including 5 or 6 carbon atoms, and p stands for numbers from 1 to 10; and (iv) the mixtures thereof.

Particularly preferred methods according to the invention are therefore characterized in that the cosmetic agent (M1) additionally includes at least one nonionic surfactant from the group comprising alkyl polyglucosides of formula R¹O-[G]ₚ, where R¹ stands for an alkyl and/or alkenyl functional group including 4 to 22 carbon atoms, G stands for a sugar functional group including 5 or 6 carbon atoms, and p stands for numbers from 1 to 10, in an overall quantity of 0.1 to 10% by weight, preferably 0.5 to 5% by weight, in particular 0.8 to 3% by weight, based on the total weight of the composition (M1). In the formula R¹O-[G]ₚ, the index number p indicates the degree of oligeromerization (DP), i.e., the distribution of mono- and oligoglucosides, and stands for a number between 1 and 10. While p in a given compound must always be an integer, and may primarily assume the values p=1 through 6 here, the value p for a specific alkyl oligoglucoside is an analytically determined mathematical variable which usually represents a fractional number. Alkyl and/or alkenyl oligoglucosides having an average degree of oligeromerization p of 1.1 to 3.0 are preferably used according to the invention. From an application standpoint, alkyl and/or alkenyl oligoglucosides are preferred whose degree of oligeromerization is less than 1.7, in particular between 1.2 and 1.7. The alkyl or alkenyl functional group R¹ may be derived from primary alcohols including 4 to 20, preferably 8 to 16, carbon atoms. Very particularly preferred according to the invention are alkyl oligoglucosides based on hydrogenated C_{12/14} coco alcohol having a DP of 1 to 3, as are commercially available, for example, under the INCI name “Coco Glucoside.” For example, addition products of 20 to 60 moles of ethylene oxide with castor oil and hydrogenated castor oil, in particular the compounds known under the INCI names PEG-40 Hydrogenated Castor Oil and PEG-60 Hydrogenated Castor Oil, are also suitable nonionic surfactants.

Furthermore, the aqueous compositions (M1) may additionally include at least one anionic surfactant. Preferred anionic surfactants are fatty acids, alkyl sulfates, alkyl ether sulfates, and ethercarboxylic acids including 10 to 20 carbon atoms in the alkyl group and up to 16 glycol ether groups in the molecule. The anionic surfactants are used in an overall quantity of 0.1 to 45% by weight, preferably 1 to 30% by weight, in particular 1 to 15% by weight, based on the total weight of the composition (M1).

In addition, it is likewise possible for the reducing agents (M1) to additionally include at least one zwitterionic and/or amphoteric surfactant. Preferred zwitterionic surfactants are betaines, N-alkyl-N,N-dimethylammonium glycinates, N-acylaminopropyl-N,N-dimethylammonium glycinates, and 2-alkyl-3-carboxymethyl-3-hydroxyethyl imidazolines. A particularly preferred zwitterionic surfactant is known under the INCI name Cocamidopropyl Betaine. Preferred amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids. Particularly preferred amphoteric surfactants are N-cocoalkylamino propionate, cocoacylaminoethylamino propionate, and C₁₂-C₁₈ acyl sarcosine. The zwitterionic and/or amphoteric surfactants are used in an overall quantity of 0.1 to 45% by weight, preferably 1 to 30% by weight, in particular 1 to 15% by weight, based on the total weight of the composition (M1).

The composition (M1) may also include at least one cationic polymer. Cationic polymers are understood to mean polymers that have groups in the main chain and/or side chain which may be “temporarily” or “permanently” cationic. According to the invention, “permanently cationic” polymers refer to those polymers which have a cationic group, regardless of the pH of the agent. These are generally polymers that include a quaternary nitrogen atom, for example in the form of an ammonium group. Quaternary ammonium groups are preferred cationic groups. In particular, those polymers in which the quaternary ammonium group is bound via a C₁₋₄ hydrocarbon group to a polymer main chain composed of acrylic acid, methacrylic acid, or the derivatives thereof have proven to be particularly suitable.

Particularly preferred cationic polymers are selected from the compounds with the INCI name “Polyquaternium.” Polyquaternium-2, Polyquaternium-4, Polyquaternium-5, Polyquaternium-6, Polyquaternium-7, Polyquaternium-10, Polyquaternium-11, Polyquaternium-17, Polyquaternium-18, Polyquaternium-22, Polyquaternium-27, Polyquaternium-37, and Polyquaternium-39 are particularly preferably used; Polyquaternium-22, Polyquaternium-37, and Polyquaternium-39 are extremely preferred, and Polyquaternium-22 is most preferred.

The following are examples of additional preferred cationic polymers:
quaternized cellulose derivatives, as are commercially available under the names Celquat® and Polymer JR®. The compounds Celquat® H 100, Celquat® L 200, and Polymer JR® 400 are preferred quaternized cellulose derivatives,
cationized honey, for example the commercial product Honeyquat® 50,
cationic guar derivatives, such as in particular the products marketed under the trade names Cosmedia® Guar and Jaguar®,
polysiloxanes having quaternary groups, for example the commercially available products Q2-7224 (manufacturer: Dow Corning; a stabilized trimethylsilylamodimethicone), Dow Corning® 929 Emulsion (containing a hydroxylamino-modified silicone, also referred to as amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, Quaternium-80),
polymeric dimethyldiallyl ammonium salts and the copolymers thereof with esters and amides of acrylic acid and methacrylic acid. The products commercially available under the names Merquat®100 (poly(dimethyldiallyl ammonium chloride)) and Merquat® 550 (dimethyldiallyl ammonium chloride-acrylamide copolymer) are examples of such cationic polymers,
copolymers of vinylpyrrolidone with quaternized derivatives of dialkylaminoalkyl acrylate and methacrylate, such as vinylpyrrolidone-dimethylaminoethyl methacrylate copolymers quaternized with diethyl sulfate, for example. Such compounds are commercially available under the names Gafquat® 734 and Gafquat® 755,
vinylpyrrolidone-vinylimidazolium methochloride copolymers, as marketed under the names Luviquat® FC 370, FC 550, FC 905, and HM 552,
quaternized polyvinyl alcohol.

Likewise usable according to the invention are the copolymers of vinylpyrrolidone, such as those available as the commercial products Copolymer 845 (manufacturer: ISP), Gaffix® VC 713 (manufacturer: ISP), Gafquat® ASCP 1011, Gafquat® HS 110, Luviquat® 8155, and Luviquat® MS 370.

The cationic polymers are included in the composition (M1) in an overall quantity of 0.1 to 5.0% by weight, in particular 0.25 to 3.0% by weight, based on the total weight of the composition (M1).

In another embodiment of the invention, the aqueous compositions (M1) may also include protein hydrolysates and/or the derivatives thereof. Protein hydrolysates are product mixtures obtained by acidic, basic, or enzymatically catalyzed degradation of proteins. Protein hydrolysates of plant and animal origin may be used according to the invention.

Cationized protein hydrolysates are usable according to the invention, wherein the underlying protein hydrolysate may originate from animals, for example from collagen, milk, or keratin, from plants, for example from wheat, corn, rice, potatoes, soybeans, or almonds, from marine life forms, for example from fish collagen or algae, or from biotechnology-derived protein hydrolysates. The protein hydrolysates on which the cationic derivatives according to the invention are based may be obtained from the corresponding proteins by chemical, in particular alkaline or acidic, hydrolysis, by enzymatic hydrolysis, and/or by a combination of the two types of hydrolysis. The hydrolysis of proteins generally results in a protein hydrolysate having a molecular weight distribution of approximately 100 Dalton all the way to several thousand Dalton. Cationic protein hydrolysates whose underlying protein component has a molecular weight of 100 to 25,000 Dalton, preferably 250 to 5000 Dalton, are preferred.

Quaternized amino acids and the mixtures thereof are also understood to be cationic protein hydrolysates. The quaternization of the protein hydrolysates or the amino acids is often carried out using quaternary ammonium salts, for example N,N-dimethyl-N-(n-alkyl)-N-(2-hydroxy-3-chloro-n-propyl)ammonium halides. In addition, the cationic protein hydrolysates may be even further derivatized. Typical examples of the cationic protein hydrolysates and derivatives according to the invention are the commercially available products listed under the INCI names in the International Cosmetic Ingredient Dictionary and Handbook, (Seventh Edition 1997, The Cosmetic, Toiletry, And Fragrance Association, 1101 17th Street, N.W., Suite 300, Washington, D.C. 20036-4702): Cocodimonium Hydroxypropyl Hydrolyzed Collagen, Cocodimonium Hydroxypropyl Hydrolyzed Casein, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxy Silicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxy Silicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, and Quaternium-79 Hydrolyzed Wheat Protein. The plant-based cationic protein hydrolysates and derivatives are particularly preferred.

The protein hydrolysates and the derivatives thereof are preferably used in an overall quantity of 0.01 to 10% by weight, based on the total weight of the composition (M1). An overall quantity of 0.1 to 5% by weight, preferably 0.1 to 3% by weight, based on the total weight of the composition (M1), is very particularly preferred.

Within the scope of the present invention, it may also be preferable for the reducing agents (M1) to include at least one oil selected from the group comprising sunflower oil, corn oil, soybean oil, pumpkin seed oil, grape seed oil, sesame oil, hazelnut oil, apricot kernel oil, orange oil, macadamia nut oil, arara oil, castor oil, avocado oil, and the mixtures thereof in an overall quantity of 0.1 to 10% by weight, preferably 0.2 to 5.0% by weight, in particular 0.5 to 2.0% by weight, based on the total weight of the cosmetic agent (M1).

The reducing agents (M1) particularly preferably include orange oil in an overall quantity of 0.001 to 1.0% by weight, preferably 0.005 to 0.5% by weight, in particular 0.01 to 0.1% by weight, based on the total weight of the composition (M1).

Thickeners may be used for thickening the aqueous composition (M1). Within the scope of the present invention, for example substances selected from cellulose ethers, xanthan gum, sclerotium gum, succinoglucans, polygalactomannans, pectins, agar, carrageenan, tragacanth, gum arabic, karaya gum, tara gum, gellan, gelatin, propylene glycol alginate, alginic acids and the salts thereof, polyvinylpyrrolidones, polyvinyl alcohols, polyacrylamides, starches that are physically modified (by pregelatinization, for example) and/or chemically modified, acrylic acid-acrylate copolymers, acrylic acid-acrylamide copolymers, acrylic acid-vinylpyrrolidone copolymers, acrylic acid-vinyl formamide copolymers, polyacrylates, and crosslinked polymers of acrylic acid or methacrylic acid and the salts thereof are suitable as thickeners. Particularly preferred thickeners are selected from cellulose ethers, in particular hydroxyalkyl celluloses. Carbomers have thickening properties.

The thickener is preferably used in the composition (M1) in an overall quantity of 0.05 to 2% by weight, in particular 0.1 to 1% by weight, based on the total weight of the composition (M1).

The composition (M2) used in method step d) is an oxidative hair dye, in particular a coloring agent, which lightens or modifies the hair color that is present prior to carrying out the method according to the invention. According to the invention, the composition (M2) used in method step d) therefore preferably includes at least one oxidation dye precursor in the form of a developer component and at least one oxidation dye precursor in the form of a coupler component. Particularly good lightening and dyeing results are obtained when oxidation dye precursors of the developer type and of the coupler type are used in the coloring agents (M2).

The developer components and coupler components are generally used in the free form. However, for substances including amino groups, it may be preferable to use their salt form, in particular in the form of the hydrochlorides and hydrobromides or the sulfates.

According to the invention, compositions (M2) are preferred which include the developer components and coupler components in each case in an overall quantity of 0.001 to 10% by weight, preferably 0.01 to 8% by weight, more preferably 0.1 to 5% by weight, in particular 0.5 to 3% by weight, based on the total weight of the composition (M2).

In another preferred embodiment, the method according to the invention is therefore characterized in that the composition (M2) used in method step d) includes the at least one oxidation dye precursor in an overall quantity of 0.001 to 10% by weight, preferably 0.01 to 8% by weight, more preferably 0.1 to 5% by weight, in particular 0.5 to 3% by weight, based on the total weight of the composition (M2).

Suitable oxidation dye precursors of the developer type are, for example, p-phenylenediamine and the derivatives thereof. Preferred p-phenylenediamines are selected from one or more compounds of the group comprising p-phenylenediamine, p-toluylenediamine, 2-chloro-p-phenylenediamine, 2,3-dimethyl-p-phenylenediamine, 2,6-dimethyl-p-phenylenediamine, N,N-bis-(2-hydroxyethyl)-p-phenylenediamine, 2-(2-hydroxyethyl)-p-phenylenediamine, 2-(1,2-dihydroxyethyl)-p-phenylenediamine, N-(2-hydroxypropyl)-p-phenylenediamine, N-(4′-aminophenyl)-p-phenylenediamine, 2-methoxymethyl-p-phenylenediamine, N-phenyl-p-phenylenediamine, 2-(2-hydroxyethyloxy)-p-phenylenediamine, and N-(4-amino-3-methyl-phenyl)-N-[3-(1H-imidazol-1-yl)propyl]amine, and the physiologically acceptable salts thereof.

It may also be preferable according to the invention to use, as the developer component, compounds that include at least two aromatic nuclei that are substituted with amino and/or hydroxyl groups. Preferred binuclear developer components are selected from N,N′-bis-(2-hydroxyethyl)-N,N′-bis-(4′-aminophenyl)-1,3-diaminopropan-2-ol, N,N′-bis-(4-aminophenyl)-1,4-diazacycloheptane, bis-(2-hydroxy-5-aminophenyl)methane, and the physiologically acceptable salts thereof.

Furthermore, it may be preferable according to the invention to use a p-aminophenol derivative or one of the physiologically acceptable salts thereof as the developer component. Preferred p-aminophenols are p-aminophenol, N-methyl-p-aminophenol, 4-amino-3-methylphenol, 4-amino-2-aminomethylphenol, 4-amino-2-(1,2-dihydroxyethyl)phenol, 4-amino-2-(diethylaminomethyl)phenol, and the physiologically acceptable salts thereof.

Moreover, the developer component may be selected from o-aminophenol and the derivatives thereof, preferably 2-amino-4-methylphenol, 2-amino-5-methylphenol, 2-amino-4-chlorophenol, and/or the physiologically acceptable salts thereof.

In addition, the developer component may be selected from heterocyclic developer components such as pyrimidine derivatives, pyrazole derivatives, pyrazolopyrimidine derivatives, and the physiologically acceptable salts thereof. Preferred pyrimidine derivatives are 2,4,5,6-tetraaminopyrimidine, 4-hydroxy-2,5,6-triaminopyrimidine, and the physiologically acceptable salts thereof. A preferred pyrazole derivative is 4,5-diamino-1-(2-hydroxyethyl)pyrazole and the physiologically acceptable salts thereof. Pyrazolo[1,5-a]pyrimidines in particular are preferred as pyrazolopyrimidines.

Preferred oxidation dye precursors of the developer type are therefore selected from the group comprising p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2, 5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol and 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, or the physiologically acceptable salts of these compounds.

Particularly preferred developer components are p-toluylenediamine, 2-(2-hydroxyethyl)-p-phenylenediamine, 2-methoxymethyl-p-phenylenediamine, N-(4-amino-3-methylphenyl)-N-[3-(1H-imidazol-1-yl)propyl]amine, and/or 4,5-diamino-1-(2-hydroxyethyl)pyrazole, and the physiologically acceptable salts thereof.

The composition (M2) used in method step d), in addition to the at least one developer component, also includes at least one coupler component as oxidation dye precursor. m-Phenylenediamine derivatives, naphthols, resorcinol and resorcinol derivatives, pyrazolones, and m-aminophenol derivatives are generally used as coupler components.

Coupler components preferred according to the invention are selected from
(A) m-aminophenol and the derivatives thereof, in particular 3-aminophenol, 5-amino-2-methylphenol, 3-amino-2-chloro-6-methylphenol, 5-amino-4-chloro-2-methylphenol, 5-(2′-hydroxyethyl)-amino-2-methylphenol, and 2,4-dichloro-3-aminophenol,
(B) o-aminophenol and the derivatives thereof, such as 2-amino-5-ethylphenol,
(C) m-diaminobenzene and the derivatives thereof, for example 2,4-diaminophenoxyethanol, 1,3-bis-(2′,4′-diaminophenoxy)propane, 1-methoxy-2-amino-4-(2′-hydroxyethylamino)benzene, 2,6-bis-(2′-hydroxyethylamino)-1-methylbenzene, 2-({3-[(2-hydroxyethyl)amino]-4-methoxy-5-methylphenyl}amino)ethanol, and 2-({3-[(2-hydroxyethyl)amino]-2-methoxy-5-methylphenyl}amino)ethanol,
(D) o-diaminobenzene and the derivatives thereof,
(E) di- or trihydroxybenzene derivatives, in particular resorcinol, 2-chlororesorcinol, 4-chlororesorcinol, 2-methylresorcinol, and 1,2,4-trihydroxybenzene,
(F) Pyridine derivatives, in particular 3-amino-2-methylamino-6-methoxypyridine, 2,6-diaminopyridine, 2, 6-dihydroxy-3,4-dimethylpyridine, 2-amino-3-hydroxypyridine, and 3,5-diamino-2,6-dimethoxypyridine,
(G) naphthalene derivatives, such as 1-naphthol and 2-methyl-1-naphthol,
(H) morpholine derivatives, such as 6-hydroxybenzomorpholine,
(I) quinoxaline derivatives,
(J) pyrazole derivatives, such as 1-phenyl-3-methylpyrazol-5-one,
(K) indole derivatives, such as 6-hydroxyindole,
(L) pyrimidine derivatives, or
(M) methylenedioxybenzene derivatives, such as 1-(2′-hydroxyethyl)amino-3,4-methylenedioxybenzene and the physiologically acceptable salts thereof.

Coupler components preferred according to the invention are therefore selected from the group comprising 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2, 6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4, 5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1, 8-dihydroxynaphthalene, 4-hydroxyindole, 6-hydroxyindole, 7-hydroxyindole, 4-hydroxyindoline, 6-hydroxyindoline, 7-hydroxyindoline, or the physiologically acceptable salts of the above-mentioned compounds.

Coupler components particularly preferred according to the invention are resorcinol, 2-methylresorcinol, 5-amino-2-methylphenol, 3-aminophenol, 2-(2,4-diaminophenoxy)ethanol, 1,3-bis-(2,4-diamino-phenoxy)propane, 1-methoxy-2-amino-4-(2′-hydroxyethylamino)benzene, 2-amino-3-hydroxypyridine, 2,6-dihydroxy-3,4-dimethylpyridine, 1,5-dihydroxynaphthalene, 2,7-dihydroxynaphthalene, and 1-naphthol, and the physiologically acceptable salts thereof.

In another embodiment, the method according to the invention is characterized in that the composition (M2) used in method step d) includes as oxidation dye precursor at least one developer component and coupler component from the group comprising p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol and 4-amino-2-(diethylaminomethyl)phenol, 4,5-diamino-1-(2-hydroxyethyl)pyrazole, 2,4,5,6-tetraaminopyrimidine, 4-hydroxy-2, 5,6-triaminopyrimidine, 2-hydroxy-4,5,6-triaminopyrimidine, or the physiologically acceptable salts of these compounds, and additionally includes at least one coupler component from the group comprising 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methyl-phenyl}amino)ethanol, 2-({3-[(2-hydroxyethyl)amino]-4, 5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1, 8-dihydroxynaphthalene, 4-hydroxyindole, 6-hydroxyindole, 7-hydroxyindole, 4-hydroxyindoline, 6-hydroxyindoline, 7-hydroxyindoline, or the physiologically acceptable salts of the above-mentioned compounds.

Oxidation dye precursors of the developer type and of the coupler type are preferably used in specific combinations. Within the scope of the present invention, the following combinations have proven to be particularly advantageous: p-toluylenediamine/resorcinol; p-toluylenediamine/2-methylresorcinol; p-toluylenediamine/5-amino-2-methylphenol; p-toluylenediamine/3-aminophenol; p-toluylenediamine/2-(2,4-diaminophenoxy)ethanol; p-toluylenediamine/1,3-bis(2,4-diaminophenoxy)propane; p-toluylenediamine/1-methoxy-2-amino-4-(2-hydroxyethylamino)benzene; p-toluylenediamine/2-amino-3-hydroxypyridine; p-toluylenediamine/1-naphthol; 2-(2-hydroxyethyl)-p-phenylenediamine/resorcinol; 2-(2-hydroxyethyl)-p-phenylenediamine/2-methylresorcinol; 2-(2-hydroxyethyl)-p-phenylenediamine/5-amino-2-methylphenol; 2-(2-hydroxyethyl)-p-phenylenediamine/3-aminophenol; 2-(2-hydroxyethyl)-p-phenylenediamine/2-(2,4-diaminophenoxy)ethanol; 2-(2-hydroxyethyl)-p-phenylenediamine/1,3-bis-(2,4-diaminophenoxy)propane; 2-(2-hydroxyethyl)-p-phenylenediamine/1-methoxy-2-amino-4-(2-hydroxyethylamino)benzene; 2-(2-hydroxyethyl)-p-phenylenediamine/2-amino-3-hydroxypyridine; 2-(2-hydroxyethyl)-p-phenylenediamine/1-naphthol; 2-methoxymethyl-p-phenylenediamine/resorcinol; 2-methoxymethyl-p-phenylenediamine/2-methylresorcinol; 2-methoxymethyl-p-phenylenediamine/5-amino-2-methylphenol; 2-methoxymethyl-p-phenylenediamine/3-aminophenol; 2-methoxymethyl-p-phenylenediamine/2-(2, 4-diaminophenoxy)ethanol; 2-methoxymethyl-p-phenylenediamine/1,3-bis(2,4-diaminophenoxy)propane; 2-methoxymethyl-p-phenylenediamine/1-methoxy-2-amino-4-(2-hydroxyethylamino)benzene; 2-methoxymethyl-p-phenylenediamine/2-amino-3-hydroxypyridine; 2-methoxymethyl-p-phenylenediamine/1-naphthol; N-(4-amino-3-methylphenyl)-N-[3-(1H-imidazol-1-yl)propyl]amine/resorcinol; N-(4-amino-3-methylphenyl)-N-[3-(1H-imidazol-1-yl)propyl]amine/2-methylresorcinol; N-(4-amino-3-methylphenyl)-N-[3-(1H-imidazol-1-yl)propyl]amine/5-amino-2-methylphenol; N-(4-amino-3-methylphenyl)-N-[3-(1H-imidazol-1-yl)propyl]amine/3-aminophenol; N-(4-amino-3-methylphenyl)-N-[3-(1H-imidazol-1-yl)propyl]amine/2-(2, 4-diaminophenoxy)ethanol; N-(4-amino-3-methylphenyl)-N-[3-(1H-imidazol-1-yl)propyl]amine/1,3-bis(2,4-diaminophenoxy)propane; N-(4-amino-3-methylphenyl)-N-[3-(1H-imidazol-1-yl)propyl]amine/1-methoxy-2-amino-4-(2-hydroxyethylamino)benzene; N-(4-amino-3-methylphenyl)-N-[3-(1H-imidazol-1-yl)propyl]amine/2-amino-3-hydroxypyridine; N-(4-amino-3-methylphenyl)-N-[3-(1H-imidazol-1-yl)propyl]amine/1-naphthol; 4,5-diamino-1-(2-hydroxyethyl)pyrazole/resorcinol; 4,5-diamino-1-(2-hydroxyethyl)pyrazole/2-methylresorcinol; 4,5-diamino-1-(2-hydroxyethyl)pyrazole/5-amino-2-methylphenol; 4,5-diamino-1-(2-hydroxyethyl)pyrazole/3-aminophenol; 4, 5-diamino-1-(2-hydroxyethyl)pyrazole/2-(2, 4-diamino-phenoxy)ethanol; 4,5-diamino-1-(2-hydroxyethyl)pyrazole/1,3-bis(2,4-diaminophenoxy)propane; 4,5-diamino-1-(2-hydroxyethyl)pyrazole/1-methoxy-2-amino-4-(2-hydroxyethylamino)benzene; 4,5-diamino-1-(2-hydroxyethyl)pyrazole/2-amino-3-hydroxypyridine; 4,5-diamino-1-(2-hydroxyethyl)pyrazole/1-naphthol. However, within the scope of the present invention it is also possible, in addition to the above-mentioned combinations, to additionally use further oxidation dye precursors in the composition (M2) used according to method step d).

Particularly attractive colorings are obtained when the composition (M2) used in method step d) includes at least one developer component selected from the group comprising p-phenylenediamine, p-toluylenediamine, N,N-bis-(2-hydroxyethyl)amino-p-phenylenediamine, 1,3-bis-[(2-hydroxyethyl-4′-aminophenyl)amino]propan-2-ol, 1,10-bis-(2′,5′-diaminophenyl)-1,4,7,10-tetraoxadecane, 4-aminophenol, 4-amino-3-methylphenol, bis-(5-amino-2-hydroxyphenyl)methane, 2,4,5,6-tetraaminopyrimidine, 2-hydroxy-4, 5,6-triaminopyrimidine, 4,5-diamino-1-(2-hydroxyethyl)pyrazole, and the physiologically acceptable salts thereof and the mixtures thereof, and at least one coupler component selected from the group comprising resorcinol, 2-methylresorcinol, 5-methylresorcinol, 2,5-dimethylresorcinol, 4-chlororesorcinol, resorcinol monomethyl ether, 5-aminophenol, 5-amino-2-methylphenol, 5-(2-hydroxyethyl)amino-2-methylphenol, 3-amino-4-chloro-2-methylphenol, 3-amino-2-chloro-6-methylphenol, 3-amino-2,4-dichlorophenol, 2,4-diaminophenoxyethanol, 2-amino-4-(2′-hydroxyethyl)aminoanisol sulfate, 1,3-bis-(2,4-diaminophenoxy)propane, 2-amino-3-hydroxypyridine, 2-methylamino-3-amino-6-methoxypyridine, 2,6-dihydroxy-3,4-dimethylpyridine, 3,5-diamino-2,6-dimethoxypyridine, 1-naphthol, 2-methyl-1-naphthol, 1,5-dihydroxynaphthalene, 2,7-dihydroxynaphthalene, 1-phenyl-3-methylpyrazol-5-one, 2,6-bis-[(2′-hydroxyethyl)amino]toluene, 4-hydroxyindole, 6-hydroxyindole, 6-hydroxybenzomorpholine, and the physiologically acceptable salts thereof and the mixtures thereof.

Furthermore, in this regard it is preferred for the composition (M2) used in method step c) to include at least one developer component selected from p-toluylenediamine and the physiologically acceptable salts thereof, and at least one coupler component selected from the group comprising resorcinol, 2-methylresorcinol, 2-amino-3-hydroxypyridine, 3-aminophenol, and the physiologically acceptable salts thereof and the mixtures thereof. When the above-mentioned oxidation dye precursors are used, particularly good lightening or dyeing is achieved which has a high level of resistance against environmental influences such as hair washing, UV light, perspiration, and abrasion.

To obtain balanced and subtle shade formation, within the scope of the present invention it may also be provided that the composition (M2) used in method step d) additionally includes at least one substantive dye. Substantive dyes are dyes that are directly absorbed onto the hair, and require no oxidative process to form the color. Substantive dyes are usually nitrophenylenediamines, nitroaminophenols, azo dyes, anthraquinones, or indophenols.

Substantive dyes may be subdivided into anionic, cationic, and nonionic substantive dyes.

Preferred anionic substantive dyes are the compounds known under the names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, and Tetrabromophenol Blue. Preferred cationic substantive dyes are cationic triphenylmethane dyes such as Basic Blue 7, Basic Blue 26, Basic Violet 2, and Basic Violet 14, aromatic systems substituted with a quaternary nitrogen group, such as Basic Yellow 57, Basic Red 76, Basic Blue 99, Basic Brown 16 and Basic Brown 17, and HC Blue 16, as well as Basic Yellow 87, Basic Orange 31, and Basic Red 51. Preferred nonionic substantive dyes are HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, and Disperse Black 9, as well as 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and the salts thereof, 2-amino-6-chloro-4-nitrophenol, 4-ethylamino-3-nitrobenzoic acid, and 2-chloro-6-ethylamino-4-nitrophenol.

In addition, naturally occurring dyes such as those included, for example, in henna red, henna neutral, henna black, chamomile blossom, sandalwood, black tea, walnut, black alder bark, sage, logwood, madder root, catechu, and alkanna root, may also be used as substantive dyes.

The composition (M2) used in method step d) preferably also includes at least one substantive dye in an overall quantity of 0.001 to 10% by weight, preferably 0.01 to 8% by weight, more preferably 0.1 to 5% by weight, in particular 0.5 to 3% by weight, based on the total weight of the composition (M2).

The oxidation dye precursors (developer and coupler) themselves are not colored. The actual dyes are not formed until the oxidation dye precursors contact an oxidizing agent (preferably hydrogen peroxide). In a chemical reaction, the developer components used as oxidation dye precursors (such as p-phenylenediamine derivatives or p-aminophenol derivatives, for example) are converted, initially oxidatively, by hydrogen peroxide into a reactive intermediate stage, also referred to as quinone imine or quinone diimine, which in an oxidative coupling reaction then reacts with the coupler components to form the particular dye.

The compositions (M2) therefore additionally include one or more oxidizing agents that are different from atmospheric oxygen. Persulfates, peroxodisulfates, chlorites, hypochlorites, and in particular hydrogen peroxide and/or one of the solid addition products thereof with organic or inorganic compounds are suitable as oxidizing agent.

Methods preferred according to the invention are therefore characterized in that the composition (M2) used in method step d) includes at least one oxidizing agent from the group comprising persulfates, chlorites, hydrogen peroxide, and addition products of hydrogen peroxide with urea, melamines, and sodium borate.

Within the scope of the present invention, it is advantageous when the composition (M2) used in method step c) includes the at least one oxidizing agent in an overall quantity of 1.0 to 12% by weight, preferably 1.5 to 12% by weight, more preferably 2.0 to 12% by weight, particularly preferably 3.0 to 12% by weight, in particular 4.0 to 12% by weight, based on the total weight of the composition (M2). This quantity of oxidizing agent ensures on the one hand sufficient fixing of the shaped keratinic fibers, and on the other hand, the reaction of the used developer components and coupler components to form the desired dyes. If hydrogen peroxide and the solid addition products thereof are used as oxidizing agent, the above-mentioned overall quantity is calculated based on 100% H₂O₂.

Hydrogen peroxide is a particularly preferred oxidizing agent within the scope of the present invention. Preferred methods according to the invention are therefore characterized in that the composition (M2) used in method step d) as oxidizing agent includes hydrogen peroxide in an overall quantity of 0.5 to 15% by weight, preferably 1 to 12.5% by weight, more preferably 1.5 to 10% by weight, in particular 1.5 to 7.5% by weight, based on the total weight of the composition (M2). The above-mentioned overall quantity is calculated based on 100% H₂O₂.

To achieve an intensified lightening and bleaching effect, the composition (M2) may also include at least one peroxo salt. Suitable peroxo salts are inorganic peroxo compounds preferably selected from the group comprising ammonium peroxodisulfate, alkali metal peroxodisulfates, ammonium peroxomonosulfate, alkali metal peroxomonosulfates, alkali metal peroxodiphosphates, and alkaline earth metal peroxides, and the mixtures thereof. Peroxodisulfates, in particular ammonium peroxodisulfate, potassium peroxodisulfate, and sodium peroxodisulfate, are particularly preferred.

The above-mentioned peroxo salts are included in an overall quantity of 0.5 to 20% by weight, preferably 1 to 12.5% by weight, more preferably 2.5 to 10% by weight, in particular 3 to 6% by weight, based on the total weight of the composition (M2).

The coloring agent (M2) may also include at least one alkalizing agent for setting a basic pH. Setting a basic pH, using the at least one alkalizing agent, is necessary to ensure opening of the outer scaly layer (cuticle) and to allow penetration of the oxidation dye precursors into the hair.

Methods preferred according to the invention are therefore characterized in that the composition (M2) used in method step d) has a pH of pH 7.0 to pH 14.0, preferably pH 8.8 to pH 11.0, more preferably pH 9.0 to pH 10.8, in particular pH 9.2 to pH 10.5, at 20° C.

Organic alkalizing agents that are usable according to the invention are preferably selected from alkanolamines of primary, secondary, or tertiary amines with a C₂-C₆ alkyl base structure bearing at least one hydroxyl group. Alkanolamines very particularly preferred according to the invention are selected from the group comprising 2-aminoethan-1-ol (monoethanolamine), 2-amino-2-methylpropan-1-ol, and 2-amino-2-methyl-propane-1,3-diol, and the mixtures thereof. Monoethanolamine is a particularly preferred alkanolamine. Suitable basic amino acids are lysine, arginine, and ornithine. Inorganic alkalizing agents according to the invention are preferably selected from the group comprising sodium hydroxide, potassium hydroxide, calcium hydroxide, barium hydroxide, sodium phosphate, potassium phosphate, sodium silicate, potassium silicate, sodium carbonate, and potassium carbonate, and the mixtures thereof.

Methods particularly preferred according to the invention are therefore characterized in that the composition (M2) used in method step d) as an alkalizing agent includes at least one compound from the group comprising sodium hydroxide, potassium hydroxide, ammonia, monoethanolamine, 2-amino-2-methylpropane, and alkali and ammonium hydrogen carbonates.

In this regard, it is particularly preferred when the composition (M2) used in method step d) as an alkalizing agent includes monoethanolamine. The occurrence of unpleasant odors during the oxidative dyeing, in particular lightening or dyeing, is thus avoided.

The alkalizing agents must be used in specific quantities in order to set a basic pH. According to the invention, the composition (M2) used in method step d) therefore advantageously includes the at least one alkalizing agent in an overall quantity of 0.1 to 15% by weight, preferably 0.5 to 12% by weight, more preferably 1.0 to 10% by weight, in particular 2.0 to 6.0% by weight, based on the total weight of the aqueous composition (M2).

The oxidative coloring agent (M2) is likewise an aqueous composition that is present in the form of a foam. The composition (M2) therefore includes at least one surfactant to ensure a stable foam.

According to one embodiment of the present invention, the composition (M2) used in method step d) as surfactant includes at least one alkyl betaine, at least one alkyl polyglucoside, and at least one nonionic surfactant. Particularly good wetting of the keratinic fibers is achieved due to the provision in the form of a foam. Particularly good wetting is necessary, since the keratinic fibers situated on the deformation aids otherwise might not be completely covered with the coloring agent (M2), leading to nonuniform dyeing results. In addition, the application as a foam prevents the coloring agent (M2) from running off during removal of the deformation aids.

In this regard, it is preferable when the composition (M2) used in method step d) includes alkyl betaines of formula (II) as surfactant:

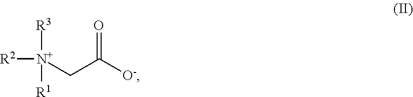

where R¹ and R³ in each case independently stand for a C₁-C₄ alkyl group or a C₂-C₄ hydroxyalkyl group, in particular a methyl group, and R² stands for a saturated or unsaturated C₁₀-C₂₀ alkyl chain, in particular a coco alkyl group. A particularly preferred alkyl betaine of formula (II) is coco alkyldimethyl betaine, for example, which is commercially available under the trade name Genagen KB from Clariant.

Furthermore, within the scope of this embodiment it is preferable when the composition (M2) used in method step d) includes alkyl polyglucosides of formula (III) as surfactant:

R⁴O-[G]ₚ (III),

where R⁴ stands for an alkyl and/or alkenyl functional group including to 22 carbon atoms, G stands for a sugar functional group including 5 or 6 carbon atoms, and p stands for numbers from 1 to 10. The index number p in general formula (III) indicates the degree of oligeromerization (DP), i.e., the distribution of mono- and oligoglucosides, and stands for a number between 1 and 10. While p in a given compound must always be an integer, and may primarily assume the values p=1 through 6 here, the value p for a specific alkyl oligoglucoside is an analytically determined mathematical variable which usually represents a fractional number. Alkyl and/or alkenyl oligoglucosides having an average degree of oligeromerization p of 1.1 to 3.0 are preferably used according to the invention. From an application standpoint, alkyl and/or alkenyl oligoglucosides are preferred whose degree of oligeromerization is less than 1.7, in particular between 1.2 and 1.7. The alkyl or alkenyl functional group R⁴ may be derived from primary alcohols including 4 to 20, preferably 8 to 16, carbon atoms. Very particularly preferred according to the invention are alkyl oligoglucosides based on hydrogenated C_{12/14} coco alcohol having a DP of 1 to 3, as are commercially available, for example, under the INCI name “Coco Glucoside.”

In addition, within the scope of this embodiment it is preferable when the composition (M2) used in method step d) includes nonionic surfactants in the form of aminoxides, in particular dimethyl cocoalkylaminoxide, as surfactant.

Moreover, within the scope of this embodiment, cationic surfactants of the quaternary ammonium compound, esterquat, or amidoamine type may also be used. Preferred quaternary ammonium compounds are ammonium halides, in particular chlorides and bromides, such as alkyl trimethyl ammonium chlorides, dialkyl dimethyl ammonium chlorides, and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride, and tricetyl methyl ammonium chloride, as well as the imidazolium compounds known under the INCI names Quaternium-27 and Quaternium-83. The long alkyl chains of the above-mentioned surfactants preferably include 10 to 18 carbon atoms.

Esterquats are known substances which include at least one ester function and at least one quaternary ammonium group as structural elements. Preferred esterquats are quaternized ester salts of carboxylic acids with triethanolamine, quaternized ester salts of carboxylic acids with diethanolalkylamines, and quaternized ester salts of carboxylic acids with 1,2-dihydroxypropyldialkylamines. Such products are marketed under the trade names Stepantex®, Dehyquart®, and Armocare®, for example. The products Armocare®VGH-70, an N,N-bis(2-palmitoyloxyethyl)dimethyl ammonium chloride, and Dehyquart®F-75, Dehyquart®C-4046, Dehyquart® L80, and Dehyquart® AU-35 are examples of such esterquats.

Alkylamidoamines are usually prepared by amidation of natural or synthetic fatty acids and fatty acid cuts with dialkylaminoamines. The stearamidopropyldimethylamine commercially available under the name Tegoamid® S 18 represents a compound from this substance group that is particularly suitable according to the invention.

The cationic surfactants are preferably used in an overall quantity of 0.05 to 10% by weight, based on the total weight of the composition (M2). An overall quantity of 0.1 to 5% by weight, based on the total weight of the composition (M2), is particularly preferred.

A large quantity of foam and a high level of foam stability may be achieved by using the above-mentioned surfactant combination. The foam-type texture of the composition (M2) results in uniform wetting of the keratinic fibers situated on the deformation aids, thus avoiding a nonuniform dyeing result. In addition, the foam-type composition (M2) may be easily distributed on the keratinic fibers, and does not run off during the exposure time.

To obtain a foam-type texture of the composition (M2), the above-mentioned surfactants must be used in a specific overall quantity. According to the invention, it is therefore preferable when the composition (M2) used in method step d) includes the at least one surfactant in an overall quantity of 5.0 to 40% by weight, preferably 10 to 35% by weight, more preferably 15 to 30% by weight, in particular 20 to 27% by weight, based on the total weight of the aqueous composition (M2).

The composition (M2) may likewise include further active substances and ingredients in addition to the above-mentioned compounds. Within the scope of another embodiment of the present invention, it may be provided that the composition (M2) used in method step d) additionally includes at least one further compound selected from the group of (i) linear or branched, saturated or unsaturated alcohols including 8 to 20 carbon atoms; (ii) cationic polymers; (iii) extracts, in particular algae extracts; and (iv) the mixtures thereof.

Within the scope of this embodiment, thickeners have proven particularly advantageous which include at least one monomer of the acrylic acid or methacrylic acid type and the derivatives thereof. A polymer very particularly preferred according to the invention is a copolymer composed of two or more monomers selected from acrylic acid, methacrylic acid, and the esters thereof with C₁-C₄ alkyl groups. A polymer very particularly preferred according to the invention is the copolymer, known under the INCI name Acrylates Copolymer, composed of two or more monomers selected from acrylic acid, methacrylic acid, and the esters thereof with C₁-C₄ alkyl groups. Another polymer very particularly preferred according to the invention is a crosslinked acrylic acid homopolymer, also referred to as a carbomer. Other very particularly preferred polymers according to the invention are methacrylic acid-free copolymers of acrylic acid and acrylic acid C₁-C₄ esters.

The thickener is preferably used in the composition (M2) in an overall quantity of 0.05 to 2% by weight, in particular 0.1 to 1% by weight, based on the total weight of the composition (M2).

Within the scope of the present invention, it may be advantageous to also add at least one linear or branched, saturated or unsaturated alcohol to the composition (M2). Alcohols including C₈-C₂₂, in particular C₁₂-C₂₂, alkyl groups, may be used as alcohols. For example, decanol, octanol, octenol, dodecenol, decenol, octadienol, dodecadienol, decadienol, oleyl alcohol, erucic alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol, and behenyl alcohol, as well as the guerbet alcohols thereof, are usable within the meaning of the invention. The alcohols preferably originate from natural carboxylic acids, typically assuming production from the esters of the carboxylic acids by reduction. Also usable according to the invention are alcohol cuts produced by reduction of naturally occurring triglycerides such as beef tallow, palm oil, peanut oil, rapeseed oil, cottonseed oil, soybean oil, sunflower oil, and linseed oil, or from the transesterification products thereof with corresponding carboxylic acid esters of appropriate alcohols, and which thus represent a mixture of different alcohols. Such substances are commercially available, for example, under the names Stenol®, for example Stenol® 1618, or Lanette®, for example Lanette® O, or Lorol®, for example Lorol® C8, Lorol® C14, Lorol® C18, Lorol® C₈₋₁₈, HD-Ocenol®, Crodacol®, for example Crodacol® CS, Novol®, Eutanol® G, Guerbitol® 16, Guerbitol® 18, Guerbitol® 20, Isofol® 12, Isofol®16, Isofol® 24, Isofol® 36, Isocarb® 12, Isocarb® 16, or Isocarb®.

The alcohols may be included in the composition (M2) in an overall quantity of 0.1 to 20% by weight, in particular 0.1 to 10% by weight, based on the total weight of the composition (M2).

The composition (M2) may also include at least one cationic polymer Suitable cationic polymers and the quantities thereof have already been discussed in conjunction with the composition (M1).

Lastly, the composition (M2) may also include at least one extract, in particular an algae extract. Algae extracts include components such as carotenoids, proteins and amino acids, polyphenols, unsaturated fatty acids and vitamins, polysaccharides, mineral substances having an antioxidant effect such as selenium and zinc, enzymes having an antioxidant effect such as catalase, superoxide dismutase, and peroxidase, as well as mineral substances such as potassium, magnesium, and iron. Aqueous or aqueous-alcohol extracts of the blue algae taxon Spirulina are algae extracts that are particularly preferably used. Such extracts may be obtained, for example, by extracting dried blue algae with water or a water/glycol mixture.

The extracts may be included in the composition (M2) in an overall quantity of 0.0001 to 1% by weight, preferably 0.001 to 0.5% by weight, in particular 0.005 to 0.1% by weight, based on the total weight of the composition (M2).

The present invention is outlined in particular by the following items:
1. A method for the permanent shaping and color modification of keratinic fibers in a single process, the method comprising the following method steps in the stated sequence:
   (a) deforming keratinic fibers, using deformation aids,
   (b) applying an aqueous composition (M1), including at least one keratin-reducing compound and at least one alkalizing agent, to the keratinic fibers situated on the deformation aids, and leaving this composition (M1) on the keratinic fibers, situated on the deformation aids, for a period of 5 to 50 minutes,
   (c) rinsing the keratinic fibers situated on the deformation aids,
   (d) applying a composition (M2), including at least one oxidation dye precursor, at least one oxidizing agent, at least one alkalizing agent, and at least one surfactant, as foam from an applicator to the keratinic fibers situated on the deformation aids, and leaving this composition (M2) on the keratinic fibers, situated on the deformation aids, for a period of 10 to 45 minutes,
   (e) removing the deformation aids from the keratinic fibers and rinsing the keratinic fibers, and
   (f) optionally applying an aftertreatment agent to the keratinic fibers.
2. The method according to item 1, characterized in that a permanent wave is carried out as permanent shaping, and lightening or dyeing is carried out as color modification.
3. The method according to one of items 1 or 2, characterized in that the deformation aids used in method step a) have a diameter of 1 to 10 cm, preferably 1 to 8 cm, more preferably 1 to 6 cm, in particular 2 to 5 cm.
4. The method according to one of the preceding items, characterized in that the composition (M1) used in method step b) includes the at least one keratin-reducing compound in an overall quantity of 5 to 20% by weight, preferably 7 to 18% by weight, more preferably 9 to 16% by weight, in particular 10 to 15% by weight, based on the total weight of the aqueous composition (M1).
5. The method according to one of the preceding items, characterized in that the composition (M1) used in method step b) includes as alkalizing agent at least one compound from the group comprising sodium hydroxide, potassium hydroxide, ammonium hydroxide, ammonia, monoethanolamine, 2-amino-2-methylpropane, and alkyl and ammonium hydrogen carbonates.
6. The method according to item 5, characterized in that the composition (M1) used in method step b) includes sodium hydrogen carbonate and/or monoethanolamine as alkalizing agent.
7. The method according to one of the preceding items, characterized in that the composition (M1) used in method step b) includes the at least one alkalizing agent in an overall quantity of 0.1 to 15% by weight, preferably 0.5 to 12% by weight, more preferably 1.0 to 10% by weight, in particular 1.5 to 7% by weight, based on the total weight of the aqueous composition (M1).
8. The method according to one of the preceding items, characterized in that the composition (M1) used in method step b) has a pH of 5 to 12, preferably 5 to 10, in particular 5 to 9.5, at 20° C.
9. The method according to one of the preceding items, characterized in that the composition (M1) used in method step b) has a weight ratio of the keratin-reducing compound to the alkalizing agent of 1:200 to 1:1, preferably 1:50 to 1:1, more preferably 1:30 to 1:1, particularly preferably 1:20 to 1:1, in particular 1:10 to 1:1.
10. The method according to one of the preceding items, characterized in that the composition (M2) used in method step d) includes the at least one oxidation dye precursor in an overall quantity of 0.001 to 10% by weight, preferably 0.01 to 8% by weight, more preferably 0.1 to 5% by weight, in particular 0.5 to 3% by weight, based on the total weight of the composition (M2).
11. The method according to one of the preceding items, characterized in that the composition (M2) used in method step d) includes, as oxidizing agent, hydrogen peroxide in an overall quantity of 0.5 to 15% by weight, preferably 1 to 12.5% by weight, more preferably 1.5 to 10% by weight, in particular 1.5 to 7.5% by weight, based on the total weight of the composition (M2).
12. The method according to one of the preceding items, characterized in that the composition (M2) used in method step d) includes as alkalizing agent at least one compound from the group comprising sodium hydroxide, potassium hydroxide, ammonia, monoethanolamine, 2-amino-2-methylpropane, and alkali and ammonium hydrogen carbonates.
13. The method according to one of the preceding items, characterized in that the composition (M2) used in method step d) includes the at least one alkalizing agent in an overall quantity of 0.1 to 15% by weight, preferably 0.5 to 12% by weight, more preferably 1.0 to 10% by weight, in particular 2.0 to 6.0% by weight, based on the total weight of the aqueous composition (M2).
14. The method according to one of the preceding items, characterized in that the composition (M2) used in method step d) as surfactant includes at least one alkyl betaine, at least one alkyl polyglucoside, at least one nonionic surfactant, and at least one anionic surfactant.
15. The method according to one of the preceding items, characterized in that the composition (M2) used in method step d) includes the at least one surfactant in an overall quantity of 5.0 to 40% by weight, preferably 10 to 35% by weight, more preferably 15 to 30% by weight, in particular 20 to 27% by weight, based on the total weight of the aqueous composition (M2).

The following examples are intended to explain preferred embodiments of the invention, without, however, limiting same.

### EXAMPLES

1. Aqueous Composition (M1)—Waving Agent

The aqueous composition (M1) in the form of a waving agent was obtained by mixing the components listed below.

TABLE 1
Waving agent

| | Quantity (% by |
|---|---|
| Raw material | weight) |
| Natrosol HR 250¹ | 0.3 |
| Monoethanolamine thioglycolate, 83% | 18 |
| Monoethanolamine | 1.3 |
| Sodium hydrogen carbonate | 2.8 |
| HEDP, 60% | 0.1 |
| Plantacare 2000 UP² | 4.0 |
| Orange oil, sweet | 0.05 |
| Gluadin W 40 BP³ | 0.1 |
| Polyquaternium-6 | 0.1 |
| Calendula KBA Herabsec⁴ | 0.1 |
| Fragrance | 0.5 |
| Water | To make 100 |

¹Natrosol HR 250 (INCI name: Hydroxyethylcellulose; Ashland)
²Plantacare 2000 UP (INCI name: Decyl Glucoside, Aqua (water); BASF)
³Gluadin W 40 BP (INCI name: Hydrolyzed Wheat Protein; BASF)
⁴Calendula KBA Herabsec (INCI name: Maltodextrin, Calendula Officinalis Flower Extract; Lipoid)

2. Oxidative Coloring Agent (M2)

The color cream described below in Table 2 was prepared, and immediately prior to application was in each case mixed in a 1:1 ratio with the oxidizing agent preparation 01 listed in Table 3:

TABLE 2
Color cream

| | Quantity (% by |
|---|---|
| Raw material | weight) |
| Plantacare 818 UP¹ | 25 |
| Genagen KB² | 30 |
| Cremophor CO 60³ | 3.0 |
| EDTA, tetrasodium salt | 0.2 |
| Monoethanolamine | 6.0 |
| Sodium sulfite | 0.2 |
| Vitamin C | 0.05 |
| L-Serine | 1.0 |
| Eau Vitale d'algue bleue⁴ | 2.0 |
| Merquat 281⁵ | 3.0 |
| p-Toluylenediamine sulfate | 1.6 |
| Resorcinol | 0.4 |
| 2-Methylresorcinol | 0.4 |
| 3-Aminophenol | 0.05 |
| 2-amino-3-hydroxypyridine | 0.03 |
| Fragrance | 0.5 |
| Water | To make 100.0 |

¹Plantacare 818 UP (INCI name: Coco Glucoside, Aqua (water), Cognis)
²Genagen KB (INCI name: Coco Betaine, Clariant)
³Cremophor CO 60 (INCI name: PEG-60 Hydrogenated Castor Oil, BASF)
⁴Eau Vitale d algue bleue (INCI name: Aqua (water), Plankton Extract, Phenoxyethanol; Soliance)
⁵Merquat 281 (INCI name: Polyquaternium-22; Lubrizol)

TABLE 3
Oxidizing agent preparation O1

| Raw material | % by weight |
|---|---|
| 50% NaOH | 0.1 |
| Polyquaternium-6 | 0.2 |
| Dehyquart A CA¹ | 0.3 |
| HEDP, 60% | 1.7 |
| Aromox MCD W² | 3.0 |
| Hydrogen peroxide, 50% | 4.0 |
| Water | To make 100.0 |

¹Dehyquart A CA (INCI name: Aqua (water), Cetrimonium Chloride; BASF)
²Aromox MCD W (INCI name: Cocamine Oxide, Akzo Nobel)

3. Experimental Procedure and Evaluation of Results:

Undamaged hair was moistened with water and rubbed with a towel. A strand of hair was then divided off in the width of the curlers used, and combed out straight. The hair strand was centrally placed in a sheet of folded perm paper and wound onto curlers having a diameter of 2 to 5 cm in each case. This procedure was repeated until all the hair was wound on curlers.

The waving agent prepared according to item 1 was applied to the wound hair and left on the hair for an exposure time of 5 to 30 minutes. The wound hair was subsequently rinsed with water having a temperature of 30° C.

After rinsing out the waving agent, the foam-type oxidative coloring agent prepared according to item 2 was applied to the wound hair and left on the hair for an exposure time of 25 to 45 minutes. The hair was then rinsed with water having a temperature of 30° C., rubbed with a towel, and dried with a hair dryer.

A uniform waving and dyeing result was obtained, and the hair experienced very little or no damage due to the method according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed:

1. A method for the permanent shaping and color modification of keratinic fibers in a single process, the method comprising the following method steps in the stated sequence:
(a) deforming keratinic fibers, using deformation aids,
(b) applying an aqueous composition (M1), including at least one keratin-reducing compound and at least one alkalizing agent, to the keratinic fibers situated on the deformation aids, and leaving this composition (M1) on the keratinic fibers, situated on the deformation aids, for a period of 5 to 50 minutes,
(c) rinsing the keratinic fibers situated on the deformation aids,
(d) applying a composition (M2), including at least one oxidation dye precursor, at least one oxidizing agent, at least one alkalizing agent, and at least one surfactant, as foam from an applicator to the keratinic fibers situated on the deformation aids, and leaving the composition (M2) on the keratinic fibers, situated on the deformation aids, for a period of 10 to 45 minutes,
(e) removing the deformation aids from the keratinic fibers and rinsing the keratinic fibers, and
(f) optionally applying an aftertreatment agent to the keratinic fibers.

2. The method according to claim 1, wherein the composition (M1) used in method step b) includes the at least one keratin-reducing compound in an overall quantity of 5 to 20% by weight based on the total weight of the aqueous composition (M1).

3. The method according to claim 1, wherein the composition (M1) used in method step b) includes as alkalizing agent at least one compound selected from the group consisting of sodium hydroxide, potassium hydroxide, ammonia, monoethanolamine, 2-amino-2-methylpropane, and alkyl and ammonium hydrogen carbonates.

4. The method according to claim 1, wherein the composition (M1) used in method step b) includes the at least one alkalizing agent in an overall quantity of 0.1 to 15% by weight based on the total weight of the aqueous composition (M1).

5. The method according to claim 1, wherein the composition (M1) used in method step b) has a weight ratio of the keratin-reducing compound to the alkalizing agent of 1:200 to 1:1.

6. The method according to claim 1, wherein the composition (M2) used in method step d) includes, as oxidizing agent, hydrogen peroxide in an overall quantity of 0.5 to 15% by weight based on the total weight of the composition (M2).

7. The method according to claim 1, wherein the composition (M2) used in method step d) includes as alkalizing agent at least one compound selected from the group consisting of sodium hydroxide, potassium hydroxide, ammonia, monoethanolamine, 2-amino-2-methylpropane, and alkali and ammonium hydrogen carbonates.

8. The method according to claim 1, wherein the composition (M2) used in method step d) includes the at least one alkalizing agent in an overall quantity of 0.1 to 15% by weight based on the total weight of the aqueous composition (M2).

9. The method according to claim 1, wherein the composition (M2) used in method step d) as surfactant includes at least one alkyl betaine, at least one alkyl polyglucoside, and at least one nonionic surfactant.

10. The method according to claim 1, wherein the composition (M2) used in method step d) includes the at least one surfactant in an overall quantity of 5.0 to 40% by weight based on the total weight of the aqueous composition (M2).
